(19) United States
(12) Patent Application Publication
(10) Pub. No.: US 2008/0274476 A1
(43) Pub. Date: Nov. 6, 2008

(54) Assay Method

(76) Inventors: Dirk Beher, Bishops Stortford (GB); Huw David Lewis, Sawbridgeworth (GB); Mark Steven Shearman, Bishops Stortford (GB)

Correspondence Address: MERCK AND CO., INC, P O BOX 2000, RAHWAY, NJ, 07065-0907, US

(21) Appl. No.: 11/792,463

(22) PCT Filed: Dec. 8, 2005
(86) PCT No.: PCT/GB05/50238

§ 371 Date: Apr. 18, 2008

(30) Foreign Application Priority Data
Dec. 9, 2004 (GB) 0427023.7

(51) Int. Cl. G01N 33/573

(52) U.S. Cl. 435/7.4

(57) ABSTRACT

There is disclosed a method for determining the activity of gamma-secretase in a text subject, using a biological sample from the subject.

This invention relates to methods for determining the activity of gamma-secretase in a test subject, in particular methods utilising a biological sample collected from the living subject.

Gamma-secretase occurs in the CNS and peripherally in man and other animals. It is a complex transmembrane aspartyl protease, containing (at least) the subunits presenilin-1, nicastrin, aph-1a and pen-2, and mediates the intra-membrane proteolysis of a variety of substrates involved in cell signalling and other biochemical pathways.

One such substrate is amyloid precursor protein (APP) which (subsequent to cleavage by beta-secretase) is cleaved by gamma-secretase to release amyloid-β (Aβ), which is believed to play a key role in Alzheimer's disease (AD) (see, for example, Hardy and Selkoe, Science, 297, 353-6 (2002)). Variability in the site of the proteolysis mediated by γ-secretase results in Aβ of varying chain length, e.g. Aβ(1-38), Aβ(1-40) and Aβ(1-42). N-terminal truncations such as Aβ(4-42) are also found in the brain, possibly as a result of variability in the site of proteolysis mediated by β-secretase. For the sake of convenience, expressions such as “Aβ(1-40)” and “Aβ(1-42)” as used herein are inclusive of such N-terminal truncated variants. After secretion into the extracellular medium, Aβ forms initially-soluble aggregates which are widely believed to be the key neurotoxic agents in AD (see Gong et al, PNAS, 100 (2003), 10417-22), and which ultimately result in the insoluble deposits and dense neuritic plaques which are the pathological characteristics of AD.

Another such substrate is the Notch protein, which is central to the Notch signalling process.

Notch signalling is elicited by receptor-ligand interaction between neighbouring cells. As a result of the receptor-ligand interaction, the Notch protein undergoes intra-membrane proteolysis, by gamma-secretase, releasing an intracellular fragment which migrates to the nucleus where it modulates gene expression.

Notch signalling plays an important part in various cellular and developmental processes, including differentiation, proliferation, survival and apoptosis (Artavanis—Tsakonas et al, Science (1999), 284, 770-776). A significant body of evidence also indicates that augmented or abnormally-prolonged Notch signalling is involved in tumorigenesis (see, for example, Callahan and Egan, J. Mammary Gland Biol. Neoplasia (2004), 9, 145-163; Collins et al, Semin. Cancer Biol. (2004), 14, 357-64; Axelson, ibid. (2004), 14, 317-319; Zweidler-McKay and Pear, ibid (2004), 14, 329-340; Weng et al, Mol. Cell. Biol. (2003), 23, 655-664; and Weng et al, Science (2004), 306, 269-271).

Inhibition of gamma-secretase is therefore of considerable therapeutic interest, and numerous small molecule inhibitors have been developed, in particular with a view to treating AD (for a review, see Harrison et al, Curr. Opin. Drug Discov. Devel. (2004), 7(5), 709-719). In connection with treatment of AD, there is also interest in compounds which modify the action of gamma-secretase so as to selectively inhibit the formation of Aβ(1-42) (see, for example, WO 01/78721 and US 2002/0128319 and Weggen et al Nature, 414 (2001) 212-16; Morihara et al, J. Neurochem., 83 (2002), 1009-12; Takahashi et al, J. Biol. Chem., 278 (2003), 18644-70, and Beher et al, J. Biol Chem., 279 (2004), 43419-26).

Pre-clinical testing of such compounds in animals, and clinical testing in humans, would be greatly facilitated by the availability of a means for monitoring the efficacy of the relevant compounds in vivo. The efficacy of gamma-secretase inhibitors or modifiers is routinely monitored in vitro using cell cultures, or membrane preparations derived therefrom (see, for example, Beher et al, Biochemistry (2003), 42, 8133-8142; Li et al, PNAS (2000), 97, 6138-6143; and GB2, 296, 415). The only ex vivo assays disclosed to date involve the use of brain homogenate as a source of the active enzyme (see, for example, Pinnix et al, J. Biol. Chem, (2001), 276, 481-487). Such assays are wasteful of experimental animals and do not allow continuous monitoring of efficacy during chronic dosing of a test compound, and are of no relevance to clinical testing in humans.

There is therefore a need for an assay for γ-secretase activity that overcomes these disadvantages.

According to the invention here is provided an ex vivo assay for gamma-secretase activity in a test subject comprising:

(a) separation of cells from a biological sample from the subject;

(b) treatment of the cells obtained in (a) with detergent to provide a solubilised membrane preparation;

(c) contacting the solubilised membrane preparation with an exogenous substrate for gamma-secretase; and

(d) detecting and quantifying a cleavage product of the exogenous substrate.

In step (a), the biological sample may be of blood or of tissue, but is preferably of blood, and the cells to be separated are preferably white blood cells or peripheral blood mononuclear cells (PBMCs). Separation of the white blood cells or PBMCs (preferably PBMCs) may be carried out by known methods, e.g. by centrifuging at 1000 g in Accuspin™ tubes (see Sigma-Aldrich Accuspin™ System—Histopaque™-1077, Procedure no. A6929/A7054/A0561). Alternatively, Leucosep™ tubes, supplied by Greiner, may be used. The cells thus obtained may be frozen and stored for later analysis, if so desired.

In step (b), the preferred detergent is CHAPSO (3-[(3-cholamidopropyl)dimethylammonio]-2-hydroxypropane-sulfonic acid). In a typical procedure for providing a solubilised membrane preparation, a suspension of cells from step (a) in distilled water (1 ml per 10 ml starting volume of blood) is diluted 5-fold with buffer containing protease inhibitors (eg Complete™) and 0.5% CHAPSO, and the mixture triturated 6 times with a 23×1 gauge syringe needle then shaken for 30 minutes at 4° C. A suitable buffer comprises 50 mM MES, 0.3 mM NaCl, 10 mM MgCl₂, giving pH 7.3

Subsequent to step (b), it is advantageous to assay the protein content of the preparation (e.g. by a standard BCA assay) to facilitate the selection of equivalent quantitites of reagents in subsequent steps.

In step (c), the exogenous substrate may be any protein or peptide which undergoes cleavage by gamma-secretase to release a fragment which can be detected and quantified. A preferred exogenous substrate is C100Flag (Li et al, supra), which is a recombinant analogue of APP and produces Aβ as a cleavage product. Suitable incubation conditions are disclosed in the aforementioned Li et al reference and in Beher et al, supra.

In step (d), detection and quantification of the cleavage product may be carried out by conventional means. When the cleavage product is Aβ, Aβ(1-40) and/or Aβ(1-42) may be assayed by incubation with labelled antibodies followed by electrochemiluminescence (ECL) analysis (e.g. as described in Beher et al, J. Biol. Chem. (2001), 276, 45394-45402).

The signal thus obtained is preferably corrected by subtraction of the background signal obtained by repeating the assay, with the modification that step (c) is carried out in the presence of excess of a known gamma-secretase inhibitor. Suitable inhibitors include the compound identified as L-685,458 (Shearman et al, Biochemistry (2000), 34, 8698-8704), and compounds disclosed in WO 03/093252.

Most commonly, steps (c) and (d) are carried out using automated techniques involving multi-well plates where some of the wells contain the known inhibitor. If desired, the actual concentration of cleavage product may be determined by reference to a calibration curve generated from measurements performed on known concentrations of authentic product.

The quantity of blood required for the assay is sufficiently small that at least in the case of larger animals such as primates and canines, the assay may be carried out without sacrificing the animal. In such cases, the assay may be repeated at suitable intervals using blood from the same subject, enabling changes in enzyme activity over time to be monitored. Most importantly, the assay may be performed on blood obtained from human subjects as well as from test animals, including primates, canines and rodents (in particular rats or mice). In a preferred embodiment, the blood is obtained from subjects previously treated with a putative inhibitor or modifier of gamma-secretase, the assay thereby providing a measure of the efficiency of said inhibitor or modifier in vivo.

In one embodiment, the assay is performed on a blood sample obtained from a subject treated with a test compound which is known to inhibit the action of gamma-secretase in vitro.

In another embodiment, the assay is carried out on a blood sample obtained from a subject treated with a test compound which is known to selectively inhibit, in vitro, the formation of Aβ(1-42) by gamma-secretase mediated cleavage of APP.

The assay thus enables monitoring of the in vivo efficacy of test compounds towards peripheral gamma-secretase in test subjects. Provided that the relevant PK parameters of the test compounds are known, the results may be used to estimate efficacy towards gamma-secretase in the CNS, in particular in the brain. This may be achieved by measuring the actual levels of Aβ in the brains of test animals (using known procedures), and correlating the results with the level of enzyme inhibition in the periphery, measured by the assay described herein. Using this correlation, the degree of peripheral enzyme inhibition in human subjects caused by a test compound can provide an estimate of the degree of inhibition in the brain of said subjects, with the caveat that the brain to plasma ratio of the test compound must be estimated (e.g. by interpolation from measurements on other species).

### EXAMPLES

A typical protocol is as follows:
1. Male CD rats are anaesthetised with pentobarbitone (approx 60 mg/kg) and the ascending vena cava exposed. The animals are heparinised (approx 0.5 ml, 1000 unit/ml, i.v.), a terminal blood sample (8-12 mls) is taken from the vena cava and the animals are exsanguinated.
   2. The blood is carefully pipetted into Accuspin tubes that have previously been warmed to room temperature. Two tubes per sample are used if the volume is >6 ml.
   3. Tubes are spun at 1000 g and the white band of PBMC (peripheral blood mononuclear cells) above the is aspirated into 2 ml Eppendorfs.
   4. The PBMC are pelleted in a benchtop centrifuge for 1 min at 1000 g and the plasma aspirated off.
   5. The PBMC pellet is washed at least once by addition of 1 ml PBS, vortexed to resuspend, and pelleted another spin cycle.
   6. The washed pellet is snap-frozen at −80° C. (possible to stop for overnight step depending on timings)
   7. After thawing, the pellet is resuspended in distilled water at a ratio of 1 ml water per every 10 ml startin volume of blood.
   8. The cell suspension is then bulked up with 5 volumes 1×MES buffer (50 mM MES, 0.3 mM NaCl, 10r MgCl₂, pH7.3), 1× protease inhibitors (Complete™) and CHAPSO to 0.5%, triturated 6× with a 23×1 gauge syringe needle and solubilised by shaking at 4° C. for 30 min.
   9. After this incubation, the extracts are vortexed and [protein] determined by standard BCA assay.
   10. When all the concentrations are known, the extracts are diluted to a common concentration (typically 0-1 mg/ml) in 1×MES buffer/0.5% CHAPSO/1× protease inhibitors) and the following incubation set
      5 μl DMSO (or 20× gamma-secretase inhibitor)
      35 μl Premix (1.5 μl 20% CHAPSO, 8.5 μl water, 25 μl 4× assay buffer*)
      20 μl C100Flag substrate
      40 μl normalised membrane preps (vortexed again)
   [All extracts should be run with some wells containing DMSO and others 10 μM
   L-685458 (or equivalent) to allow the non-specific assay signal to be subtracted. Inclusion of a standard curve of synthetic peptide would also allow quantitation of the Aβ(40) generated.]
   11. After 3 hrs mixing at 37° C., 75 μl are removed for a standard overnight Aβ(40) detection assay using 4G8Bio and G2-10Ru, using ECL technology and either the Bioveris M-8 Origen machine or the Mes Scale Discovery Sector 6000 imager.
      * 4× assay buffer=80 mM HEPES, 8 mM EDTA, 0.4% BSA

### Example 1

The above protocol was followed using blood from naïve rats and beagle dogs. A robust ECL signal was obtained in each case. When known γ-secretase inhibitors were added to the wells in step 10, the signals were attenuated in a dose-dependent fashion, indicating that the PBMCs are a viable source of active enzyme.

### Example 2

Two rats were each dosed with the compound disclosed in Ex. 14 of WO 03/093252 at a dose (30 mg/Kg p.o.) calculated to provide 100% inhibition of γ-secretase in vivo. Fours hours later, blood samples were taken and processed as described above, and blood samples from two untreated controls were processed likewise. The control samples each gave similar, robust ECL signals, but no appreciable signal was detected from either of the test samples, indicating that inhibition of the enzyme survived the extraction procedure.

### Example 3

Rats were assigned to the following treatment groups (3 per group): controls (vehicle), positive controls (Ex. 14 of WO 03/093253, 30 mpk), test 1 (10 mpk test compound), test 2 (30 mpk test compound) and test 3 (100 mpk test compound). Four hours after dosing, blood samples were processed as described previously, and the results are summarised in the following table:

| Group | ECL Signal* |
|---|---|
| controls | 100% |
| +ve controls | 1% |
| test 1 | 59% |
| test 2 | 24% |
| test 3 | 21% |

*average of 3, corrected for background (non-specific) signal.

Thus, the assay detected a dose-dependent inhibition of γ-secretase in vivo. (The similar results for the test 2 and test 3 groups reflected similar plasma levels of the drug in these groups, in spite of the difference in dose).

### Example 4

To confirm that these protocols can be applied to the measurement of γ-secretase activity in primates, PBMC pellets were extracted from rhesus monkey blood and from human blood donated by three subjects, and processed as described above, and compared with rat samples. In all cases a strong signal for Aβ(40) (generated ex vivo) was detected. This signal was attenuated in a dose-dependent manner by spiking with a known γ-secretase inhibitor, whose potency (represented by the calculated IC₅₀) was shown to be broadly similar in all three species.

1. An ex vivo assay for gamma-secretase activity in a test subject comprising:
(a) separation of cells from a biological sample from the subject;
(b) treatment of the cells obtained in (a) with detergent to provide a solubilised membrane preparation;
(c) contacting the solubilised membrane preparation with an exogenous substrate for gamma-secretase; and
(d) detecting and quantifying a cleavage product of the exogenous substrate.

2. An assay according to claim 1 wherein the cells in step (b) are white blood cells or peripheral blood mononuclear cells.

3. An assay according to claim 1 wherein the exogenous substrate in step (c) is C100Flag.

4. An assay according to claim 1 wherein the cleavage product detected in step (d) is Aβ(1-40) or Aβ(1-42).

5. An assay according to claim 4 in which the cleavage product is detected and quantified using electrochemiluminescence analysis.

6. An assay according to claim 1 wherein the cells are obtained from a test subject that has been treated with a compound known to inhibit the action of gamma-secretase in vitro.

7. An assay according to claim 1 wherein the cells are obtained from a test subject that has been treated with a compound known to selectively inhibit, in vitro, the formation of Aβ(1-42) by gamma-secretase mediated cleavage of APP.

8. An assay according to claim 6 wherein the test subject is human.

9. An assay according to claim 7 wherein the test subject is human.
